(19) United States
(12) Patent Application Publication
(10) Pub. No.: US 2011/0262455 A1
(43) Pub. Date: Oct. 27, 2011

(54) TREATMENT OF PROLIFERATIVE DISORDERS WITH A DEATH RECEPTOR AGONIST

(75) Inventors: Afshin Samali, Galway (IE); Eva Szegezdi, Galway (IE); Devalingam Mahalingam, Galway (IE); Alessandro Natoni, Galway (IE)

(73) Assignee: National University of Ireland, Galway (IE)

(21) Appl. No.: 13/123,348

(22) PCT Filed: Oct. 9, 2009
(86) PCT No.: PCT/IB2009/007170

§ 371 Date: Jun. 30, 2011

(30) Foreign Application Priority Data
Oct. 10, 2008 (GB) 0818649.6

(51) Int. Cl. A61K 39/395; A61P 35/00; A61K 31/7088; A61K 38/17

(52) U.S. Cl. 424/158.1; 514/19.3; 514/19.4; 514/19.5; 514/19.6; 514/44 R; 514/44 A

(57) ABSTRACT

A method of treating a proliferative disorder, and a pharmaceutical composition for use in such a method, comprises administering to the patient a combination of an agonist of a death receptor and an antagonist of Egr-1. The death receptor agonist and the Egr-1 antagonist may be administered sequentially, separately or in combination

### FIELD OF THE INVENTION

The present invention relates to a combination therapy involving a death receptor agonist for treating a proliferative disorder. In particular, the invention relates to a method for treating proliferative disorders using a death receptor agonist and an antagonist of Egr-1.

### BACKGROUND OF THE INVENTION

Death receptor agonists are molecules which bind to death receptors and induce apoptosis or programmed cell death through a variety of intracellular pathways. These pathways generally function by bringing their cytoplasmic portions into close proximity, leading to the recruitment and activation of downstream effector proteins. Death receptors form a subclass of the Tumor Necrosis Factor Receptor (TNFR) superfamily which encompasses eight members: Fas, TNFR1, neurotrophin receptor (p75NTR), ectodysplasin-A receptor (EDAR), death receptor (DR) 3, DR4, DR5, and DR6. Amongst the most well studied death receptor agonists are members of the TNF ligand family, which can play key roles in regulatory and deleterious effects on immune tolerance, in addition to both protective and pathogenic effects on tissues (Rieux-Laucat et al., 2003, Current Opinion in Immunology 15:325; Mackay and Ambrose, 2003, Cytokine and growth factor reviews, 14: 311; Mackay and balled, 2002, Current opinion in Immunology, 14: 783-790). Examples of such proteins include Tumour necrosis factor-related apoptosis inducing ligand (TRAIL), Fas ligand (FasL) and Tumor Necrosis Factor (TNF). (Ashkenazi, A., and Dixit, V. M. (1998) Science 281, 1305-1308).

Death receptor agonists induce apoptosis upon binding to transmembrane, death domain containing receptors. For example, TRAIL binds to death receptor 4 (DR4; TRAIL receptor 1) and 5 (DR5; TRAIL receptor 2). Three other TRAIL-binding receptors exist, but are considered to be “decoy receptors” as they are unable to transmit an apoptotic signal. Decoy receptor 1 (DcR1) lacks the transmembrane and intracellular domains and is anchored to the plasma membrane via a glycosylphosphatidylinositol-tail. Decoy receptor 2 (DcR2) possesses a truncated, non-functional death domain, while the third decoy receptor, osteoprotegerin is a secreted, soluble receptor. Fas ligand induces apoptosis by binding to Fas (also known as CD95 or Apo-1), while DcR3 sequesters FasL from Fas. Another death receptor agonist, TNF can induce apoptosis by binding to TNF-receptor I (also known as TNFR1 or TNFR55).

TRAIL, in its soluble form, selectively induces apoptosis in tumour cells in vitro and in vivo. TRAIL appears to be inactive against normal healthy tissue, therefore attracting great interest as a potential cancer therapeutic (Ashkenazi et al (1999), J. Clin. Invest 104, 155-162). Therefore TRAIL has the potential to serve as a safe and potent therapeutic agent against tumour cells. Recent development also achieved specific, tumoricidal activity of other death ligands, such as FasL and TNF (as reviewed in Papenfuss et al. J Cell Mol. Med. 2008 (6B):2566-85) A number of in vitro studies have shown that many tumour cell lines of divergent origins are sensitive to TNF ligand family member induced apoptosis and especially to TRAIL induced apoptosis.

However, although TRAIL preferentially induces apoptosis in a wide variety of cancer cells, not all tumours are sensitive to TRAIL (Di Pietro et al (2004), J Cell Physiol, 201(3): p. 331-340). Growing evidence suggests that many human derived primary cancer cell lines, chronic lymphocytic leukemia (CLL), astrocytoma, meningioma and medulloblastoma, are resistant to TRAIL and other TNF ligand family members, despite the expression of the corresponding death receptors (Dyer, M. J. et al. (2007), J Clin Oncol. 25(28): p. 4505-4506). The underlying mechanism of such resistance is complex, potentially involving many, as yet, unknown mediators.

Given the benefits of TRAIL and other TNF ligand family members, it would be desirable to have TRAIL (or other death ligands) available for treatment of as many different forms of cancer as possible. There is therefore a need in the art to provide a therapy that will allow the treatment of tumours and other proliferative disorders with TRAIL or other TNF ligand family members which would otherwise not respond or not respond very well to treatment with these proteins.

### SUMMARY OF THE INVENTION

According to an embodiment of the invention there is provided a method of treating a proliferative disorder in a patient comprising administering to the patient a combination of a an agonist of a death receptor and an antagonist of Egr-1, wherein said death receptor agonist and said Egr-1 antagonist may be for sequential, separate or combined administration.

In certain embodiments the death receptor agonist may be a Tumour Necrosis Factor (TNF) family member. In particular embodiments the death receptor agonist may be TRAIL, Tumor Necrosis Factor (TNF), Fas ligand, or a variant thereof.

In certain embodiments of the invention the proliferative disorder is characterized by at least 1.5-fold increased expression of Egr-1 in cells affected by the proliferative disorder compared to the expression levels of Egr-1 in cells unaffected by the proliferative disorder from the same subject. In a specific embodiment the proliferative disorder is cancer. Said cancer may be selected from the group consisting of cancers of the lung, breast, prostate, bladder, kidney, ovaries, colon, rectal, melanoma, leukemia, multiple myeloma and gynecological cancers.

In other embodiments of the invention, the Egr-1 antagonist is selected from the group consisting of antibodies, dominant negative Egr-1 variant expressing vectors, peptides, small molecule inhibitors, RNAi (shRNA, shRNA expressing vectors, siRNA), microRNA (miRNA) and so on. The invention also provides a pharmaceutical composition comprising a death receptor agonist such as a Tumor Necrosis Factor ligand family member including TRAIL, TNF or Fas ligand and an antagonist of Egr-1, as well as a death receptor agonist such as a Tumor Necrosis Factor ligand family member including TRAIL, TNF or Fas ligand and an antagonist of Egr-1 for treating a proliferative disorder.

In certain embodiments the death receptor agonist may be a death receptor agonist variant, such as, for example, a TRAIL variant, a TNF variant or a fas ligand variant.

For example, a TRAIL variant may have substantially greater affinity for the death receptor 4 (TRAIL-R1) over its affinity for the death receptor 5 (TRAIL-R2). In another example, it is envisioned to use a TRAIL variant that has substantially greater affinity for the death receptor 5 (TRAIL-R2) over its affinity for the death receptor 4 (TRAIL-R1). The TRAIL variant may also have substantially greater affinity for the death receptor 4 (TRAIL-R1) and/or the death receptor 5 (TRAIL-R2) over its affinity for the decoy receptor DcR1 (TRAIL-R3) and/or DcR2 (TRAIL-R4). In certain embodiments the death receptor agonist variant may be a TRAIL variant and the TRAIL variant may be selected from the group consisting of G131R, G131K, R1491, R149M, R149N, R149K, S159R, Q193H, W193K, N199R/K201H, N199H/K201R, G131R/N199R/K201H, G131R/N199R/K201H, G131R/D218H, K201R, K204E, K204D, K204L, K204Y, K212R, S215E, S215H, S215K, S215D, D218H, K251D, K251E, K251Q, D269H, E195R, D269H/E195R, T214R and D269H/T214R.

As a further example, the death receptor agonist variant may be a TNF variant. The TNF variant may be selective for TNFR-I (TNFR55), such mutants may include R32W, R32W-S86T, or E146K.

The death receptor agonist variant may be a Fas ligand variant. The fas ligand variant may have increased affinity for Fas and may vary at one or more positions from wild type Fas ligand.

### DETAILED DESCRIPTION OF THE PREFERRED EMBODIMENTS

### Definitions

As used herein, the following terms and phrases shall have the meanings set forth below. Unless defined otherwise, all technical and scientific terms used herein have the same meaning as commonly understood by one of ordinary skill in the art to which this invention belongs.

The articles “a” and “an” refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used herein to mean “including but not limited to”. “Including” and “including but not limited to” are used interchangeably.

A “patient”, “subject” or “host” to be treated by the method of the invention may mean either a human or non-human animal and is preferably a mammal, more preferably a human. The human may be a child or an adult.

“Combined” administration means that the death receptor agonist and the Egr-1 antagonist are administered together, for example in the same injection device or tablet. In order for the administration to be considered “combined” the active components of the pharmaceutical composition need to be mixed. In contrast, “separate” means that the death receptor agonist and the Egr-1 antagonist are not mixed but administered separately at approximately the same time. In this regard, the skilled person will understand that it is not always practical to administer the death receptor agonist and the Egr-1 antagonist exactly simultaneously. Therefore, a treatment is considered separate when the beginning of the administration of the death receptor agonist and the beginning of the administration of the Egr-1 antagonist fall within a time frame of 1 hour, 50 minutes, 40 minutes, 30 minutes, 20 minutes, 15 minutes, 10 minutes or 5 minutes of each other.

“Sequential” administration refers to any administration which is not considered combined or separate, as defined above. The death receptor agonist and the Egr-1 antagonist may thereby be administered in any order. For example, the Egr-1 antagonist may be administered before the death receptor agonist. If the Egr-1 antagonist is administered first, the death receptor antagonist needs to be administered while the Egr-1 antagonist still exerts its biological effect. Depending on the type of Egr-1 antagonist used, this may mean that the Egr-1 protein is not expressed or is expressed at a lower level, compared to cells which have not been treated with the Egr-1 antagonist. If, for example, the Egr-1 antagonist acts by repressing transcription of the Egr-1 gene, the antagonist would be considered to exert its biological effect when transcription of the Egr-1 gene in cells which were treated with the antagonist is reduced by 5%, 10%, 15%, 20% 30%, 40%, 50%, 60%, 70%, 80%, 95%, 99% or even 100% compared to cells which were not exposed to the Egr-1 antagonist, as determined by standard techniques such as real-time PCR on cDNA etc. It may also mean that Egr-1 does not exert its biological function by a reporter assay or electromobility shift assay. If the death receptor agonist is administered first, the Egr-1 antagonist preferably needs to be administered while the death receptor agonist is still detectable in the blood of the treated subject by standard biological methods, such as Western blotting or Elisa.

As used herein, an “antagonist” is a molecule which interferes with the biological function of a protein. The antagonist may thereby bind to the target protein to elicit its functions. However, antagonists which do not bind the protein are also envisioned. The antagonist may inhibit the biological function of the protein directly or indirectly. Examples of antagonists which may interfere with protein function are dominant negative mutants of the protein (for example mutants lacking the functional domain), small molecules, synthetic or native sequence peptides, native sequence peptides or antibodies. Antagonists which inhibit or reduce expression of a gene encoding for the Egr-1 protein are also envisioned and are within the scope of the invention. Examples of such antagonists are siRNAs, miRNAs, small molecules etc. Other suitable antagonists will be evident to those of skill in the art. For the present invention it is not necessary that the antagonist interferes with the function of the Egr-1 protein directly. Rather, antagonists are also envisioned which interfere with the function of one or more proteins upstream in a pathway that eventually leads to activation of Egr-1. For example, Egr-1 is known to be transcriptionally regulated by Elk-1, an ETS-domain transcription factor, activated by hypoxia (Yan S F, et al. (1999); J Biol. Chem. May 21; 274(21):15030-40.) which may make it possible to target Elk-1, or it's upstream activator, PKCβII (protein kinase beta II) in order to reduce or suppress the biological function of Egr-1.

In contrast, an “agonist” as used herein is a molecule which enhances the biological function of a protein. The agonist may thereby bind to the target protein to elicit its functions. However, agonists which do not bind the protein are also envisioned. The agonist may enhance the biological function of the protein directly or indirectly. Agonists which increase expression of certain genes are envisioned within the scope of particular embodiments of the invention. Suitable agonists will be evident to those of skill in the art. For the present invention it is not necessary that the agonist enhances the function of the target protein directly. Rather, agonists are also envisioned which stabilize or enhance the function of one or more proteins upstream in a pathway that eventually leads to activation of Egr-1. Alternatively, the agonist may inhibit the function of a negative transcriptional regulator of the target protein, wherein the transcriptional regulator acts upstream in a pathway that eventually represses transcription of the target protein.

“Death receptors” form a subclass of the Tumor Necrosis Factor Receptor (TNFR) superfamily which encompasses eight members: Fas, TNFR1, neurotrophin receptor (p75NTR), ectodysplasin-A receptor (EDAR), death receptor (DR) 3, DR4, DR5, and DR6. Most of the death receptors have their corresponding natural ligands identified: TNFR1 can be activated by TNF, Fas is activated by Fas ligand, p75NTR is activated by nerve growth factor (NGF, gene ID: 4803). One ligand for EDAR is ectodysplasin-A (EDA, gene ID: 1896). DR3 can be activated by Apo3L (TWEAK/TNFSF12, gene ID: 8742), TL1A/VEGI (vascular endothelial growth inhibitor/TNFSF15, gene ID: 9966), while DR4 and DR5 share the same ligand, TNF-related apoptosis-inducing ligand (TRAIL). The ligand for DR6 has not been identified. These ligands, their variants or any molecule that mimic the effect of the natural ligand is considered as a death receptor agonist. Each of these natural ligands and agonists thereof is considered a death receptor agonist.

A “death receptor agonist” is defined as any molecule which is capable of inducing pro-apoptotic signaling through one or more of the death receptors. The death receptor agonist may be selected from the group consisting of antibodies, death ligands, cytokines, death receptor agonist expressing vectors, peptides, small molecule agonists, cells (for example stem cells) expressing the death receptor agonist, and drugs inducing the expression of death ligands.

A “Tumor Necrosis Factor family member” or a “Tumor Necrosis Factor ligand family member” is any cytokine which is capable of activating a Tumor Necrosis Factor receptor.

“TRAIL protein”, as used herein, encompasses both the wt TRAIL protein and TRAIL variants.

By “variant” death receptor agonist it is meant that the death receptor agonist differs in at least one amino acid position from the wild type sequence of the death receptor agonist.

By “variant” TRAIL protein it is meant that the TRAIL protein differs in at least one amino acid position from the wild type TRAIL protein (also known as TNFSF10, TL2; APO2L; CD253; Apo-2L), Entrez GeneID: 8743; accession number NM_{—}003810.2; UniProtKB/Swiss-Prot: P50591; UniProtKB/TrEMBL: Q6IBA9.

By “variant” Tumor Necrosis Factor protein it is meant that the Tumor Necrosis Factor protein differs in at least one amino acid position from the wild type Tumor Necrosis Factor protein (also known as TNF; DIF; TNF-alpha; TNFA; TNFSF2), accession number NM_{—}000594.

By “variant” Fas ligand protein it is meant that the Fas ligand protein differs in at least one amino acid position from the wild type Fas ligand protein (also known as FASLG; APT1LG1; CD178; CD95L; FASL; TNFSF6), accession number NM_{—}000639.

“Apoptosis rate” is the percentage of cells in a sample which are undergoing or have undergone apoptosis in relation to the total number of cells in a sample. There are numerous assays available which will allow the skilled person to establish the apoptosis rate, for example Annexin V staining.

### Treatment Methods

In one embodiment, the invention relates to a method for treating a proliferative disorder in a patient comprising administering to the patient a combination of a death receptor agonist and an antagonist of Egr-1, wherein said death receptor agonist and said antagonist are for sequential, separate or combined administration.

In one embodiment said death receptor agonist may be a TNF family member. In a further embodiment, said death receptor agonist may be TRAIL, TNF or Fas ligand.

One problem with the use of death receptor agonists, and particularly TRAIL to treat tumours is that certain tumours are resistant to the effects of death receptor agonists including TRAIL, despite expression of DR4 and DR5 receptors on the cell surface. Using microarray experiments, the inventors have discovered that Egr-1 is upregulated in response to treatment of cells with TRAIL and that the upregulation of Egr-1 in the cell results in upregulation of c-FLIP, an anti-apoptotic protein which inhibits pro-caspase-8 activation. This discovery led the inventors to hypothesize that antagonizing Egr-1 would increase the apoptosis rate as c-FLIP would also be inhibited. Using a dominant negative mutant that lacks the trans activation domain found in wt Egr-1, the inventors have now demonstrated that inhibition of Egr-1 does indeed result in an increased apoptosis rate following treatment with TRAIL (FIG. 2).

The inventors have also discovered that Egr-1 increases resistance of tumour cells against the death ligands Fas ligand and TNF. This lead the inventors to further hypothesise that antagonizing Egr-1 would increase the apoptotic rate by reducing the cell's resistance to all death ligands belonging to the TNF ligand superfamily, including Fas ligand and/or TNF, and thereby increasing Fas ligand- and/or TNF-mediated apoptosis.

This increase in apoptosis rate, following treatment with the Egr-1 antagonist, will allow treatment of tumours which are otherwise resistant to the effects of death receptor agonists. Furthermore, it may also increase the efficiency of conventional TRAIL treatment as tumours are likely to respond better to death receptor agonists, and particularly to TRAIL. Therefore, the inventors' discovery is likely to have wide implications in future therapy of proliferative disorders using death receptor agonists and particularly to TRAIL-based therapies.

The gene encoding Early Growth Response-1 (Egr-1) protein, which is also known as NGFI-A, zif268, krox24 and Tis8, is an immediate-early gene encoding a Cys2-His2-type zinc-finger transcription factor. It is rapidly activated by multiple extracellular agonists (such as growth factors and cytokines) and environmental stresses (such as hypoxia, fluid shear stresses, and vascular injury). Egr-1 induces or represses its target genes by preferentially binding to GC-rich regulatory elements. Egr-1 is important in regulating cell growth, differentiation, and development.

### Antagonists of Egr-1 Expression

There are a variety of antagonists of Egr-1 which can be used with the present invention. The antagonist may be a transcriptional antagonist or a functional antagonist and may affect transcription of Egr-1 or the biological function of Egr-1 either directly or indirectly. It is also envisioned to use a combination of two or more inhibitors of Egr-1 in the method of the present invention.

In one embodiment the Egr-1 antagonist prevents or reduces transcription of the Egr-1 gene. Suitable antagonists may include siRNAs, shRNA expression vectors or miRNAs. A transcriptional antagonist is thereby considered suitable for use in the invention if it reduces transcription of the Egr-1 gene by at least 5%, 10%, 15%, 20%, 25%, 30%, 35%, 40%, 45%, 50%, 55%, 60%, 65%, 70%, 75%, 80%, 85%, 90%, 95%, 99% or even 100% compared to the transcription of the Egr-1 gene in cells which were not treated with the antagonist. It is thereby not necessary that a particular antagonist represses transcription of Egr-1 directly but it is also possible to use an antagonist that inhibits transcription by affecting the transcription or biological function of a transcriptional regulator of Egr-1 that acts upstream of Egr-1, such as Elk-1. Methods of determining whether Egr-1 shows reduced transcription are well known to those of skill in the art and include but are not limited to real-time polymerase chain reaction (RT-PCR) using Egr-1 specific primers on cDNA extracted from a cell, semi-quantitative PCR, Northern Blotting, electromobility shift assay or reporter assay (Yan S F et al. (1999); J Biol. Chem.; 274(21):15030-40). Where appropriate, it may be desirable to check that decreased transcription concurs with decreased protein levels in the cells. Suitable techniques to confirm this will be evident to those of skill in the art and include, but are not limited to, Western blot analysis, ELISA, etc.

In one embodiment the antagonist is a siRNA molecule. Examples of siRNA sequences suitable for use in the present invention are shown in Table 3. Bioinformatics tools suitable for designing other siRNA molecules suitable to suppress transcription of Egr-1 will be known to those of skill in the art. One example of such a tool is the siRNA selection program provided by the Whitehead Institute, Cambridge (http://jura.wi.mit.edu/bioc/siRNAext/).

TABLE 3

| | |
|---|---|
| UUGGGCCAAUGAUGGAGAAUA | SEQ ID NO. 1 |
| UUCACGACGAUAGUUAAUAAU | SEQ ID NO. 2 |
| UUCGAGGGAAGUUACGAUCUU | SEQ ID NO. 3 |
| UUCACUGACAAACCGAAUAUU | SEQ ID NO. 4 |
| UUGAGGGAAGUUACGAUCUUU | SEQ ID NO. 5 |
| UUCCAGGUCCUUGUAACGUUA | SEQ ID NO. 6 |

Suitable methods to screen for a transcriptional antagonist will be evident to those of skill in the art. As an example, cells in culture could be exposed to an agent, which is tested for its ability to reduce or inhibit transcription of Egr-1. In parallel, the same type of cells would be exposed to a substance which is known not to affect Egr-1 transcription (for example water), as a control. Following a suitable length of exposure, RNA would be extracted from these cells and the control cells. The expression levels of Egr-1 can then be determined by reverse transcribing the RNA extracted from the treated cells and the control cells and subjecting the obtained cDNA to real-time PCR using Egr-1 specific primers. An agent that inhibits transcription of Egr-1 by at least 5%, 10%, 15%, 20%, 25%, 30%, 35%, 40%, 45%, 50%, 55%, 60%, 65%, 70%, 75%, 80%, 85%, 90%, 95%, 99% or even 100%, when compared to the control, would be considered suitable for use in the method of the present invention.

Where an antagonist is an inhibitor of a biological function of Egr-1, it may be a dominant negative mutant of Egr-1, synthetic or native sequence peptides, antibodies, or small molecules. A mutant Egr-1 protein which lacks the trans activation domain normally found in the wt Egr-1 protein is not functional and acts as a dominant negative mutant of Egr-1 (Al-Sarraj A, et al. (2005); J Cell Biochem 94: 153-167). Given that the transactivation domain is important for the function of the protein, other antagonists, in particular small molecules, antibodies and peptides, can be screened for their ability to bind to the transactivation domain. If a molecule binds, it will prevent the transactivation domain from exercising its normal function and thereby repress the function of the protein. In a similar manner, antagonists interfering with binding of Egr-1 to its reporter element on the DNA are envisaged (small molecules, peptides, oligonucleotides and their analogues).

Therefore, one way to screen for suitable antagonists of Egr-1 function is to test for agents that bind the active domain of the protein. Such binding assays will be evident to those of skill in the art. As an example, the tested agent may be radioactively labeled using standard laboratory techniques. The thus labeled agent can then be mixed with an Egr-1 protein that was either obtained recombinantly or wildtype Egr-1 which was isolated from the cell by standard immunoprecipitation techniques. An Egr-1 protein which lacks the transactivation domain can serve as a control. The protein and the tested antagonist are then incubated for a suitable amount of time, as can be established by routine experimentation. The full length Egr-1 protein and the Egr-1 protein lacking the transactivation domain are then immunoprecipitated using an antibody that does not bind the transactivation domain of Egr-1 (for example Egr-1 (588): sc-110, Santa Cruz), washed several times and separated on an SDS gel. Binding of the antagonist can be confirmed by blotting the separated proteins on a filter using standard laboratory techniques and detecting the position of the labeled agent on the filter. If a radioactive signal can be detected at the size of the Egr-1 protein which is only present in the full-length protein, it may be assumed that the agent binds the transactivation domain of the protein. Therefore, a molecule that can bind the full-length protein but not or with less efficiency the mutant lacking the transactivation domain is suitable for use in the present invention.

It is also possible to use standard bioinformatics tools to predict which small molecules or peptides will bind the transactivation domain of Egr-1. This approach has the advantage that a large number of potential antagonists can be screened.

Antagonists which inhibit Egr-1's ability to increase resistance of tumor cells against other death receptor agonists, such as Fas ligand and/or TNF-mediated apoptosis, effectively increasing the cell's sensitivity to Fas ligand and/or TNF are also envisaged. This may be detected in any of the ways discussed above.

The antagonist may also be one which interferes with the biological function of Egr-1 without actually binding directly to the protein. Given that Egr-1 is a transcriptional regulator (Gashler A et al., (1995); Nucleic Acid Res Mol Biol; 50:191-224), it is possible to assess the biological function of the protein by monitoring the expression of downstream targets of Egr-1. By “downstream targets” we mean genes whose transcription depends either directly or indirectly on the biological function of Egr-1. Suitable assays to identify such targets will be evident to those of skill in the art.

In order to screen for an antagonist of biological function of Egr-1, one could use cells in culture stably expressing a luciferase enzyme under the control of a promoter of a gene whose transcription is dependent on the biological function of Egr-1. The cells in culture could then be exposed to an agent, which is tested for its ability to reduce or inhibit the biological function of Egr-1. In parallel, the same type of cells would be exposed to a substance which is known not to affect Egr-1 function (for example water), as a control. Following a suitable length of exposure, the cells would be lysed by standard methods, as known to those of skill in the art, and the luciferase activity in the cells and in the control cells could be assessed using standard luciferase enzyme assays (for example: Luciferase Assay kit, E1500, Promega). An agent would be considered suitable for use in the present invention if the luciferase activity in the cells treated with the agents is at least 5%, 10%, 15%, 20%, 25%, 30%, 35%, 40%, 45%, 50%, 55%, 60%, 65%, 70%, 75%, 80%, 85%, 90%, 95%, 99% or even 100% lower than in the cells which were used as a control.

The antagonists which are identified by the methods described above and other methods which will be evident to those of skill in the art can then be tested in combination with a death receptor agonist such as TRAIL, Fas ligand or TNF to see if the combination enhances apoptosis in a cell line compared to a treatment with the death receptor agonist alone. Suitable cell lines for use in such assays include, but are not limited to, human derived primary cancer cell lines, CLL, prostate cancer, astrocytoma, meningioma, colon carcinoma, Wilms' tumour and medulloblastoma cell lines, which can be obtained by standard laboratory techniques. The antagonist can be considered efficient if the apoptosis rate is increased by at least 1.5fold, 2fold, 3fold, 4fold, 5fold, 6fold, 7fold, 8fold, 9fold, 10fold, 100fold or even 1000fold by the combination of the death receptor agonist and the antagonist compared with a treatment by the death receptor agonist alone.

Apoptosis can be measured by a number of different assays, as will be clear to those of skill in the art. Examples include DNA laddering assays (see, for example, EP0835305; Immunex); detection of chromatin fragmentation and condensation with Hoechst 33342, staining and detection of phosphatidyl serine exposure in combination with membrane permeabilisation measured by staining with Annexin V and propidium iodide. What these assays share in common is a measurement of biochemical or morphological changes occurring in dying cells upon sustained contact with a concentration of the compound whose activity is being measured. Cell death can be expressed as an increase of the percentage of dying cells in response to exposure to the compound (i.e. the percentage of dying cells in untreated, control cell population is subtracted from the equivalent percentage in cell populations exposed to the drug). Alternatively, apoptosis can also be measured by caspase activation and other assays known to those of skill in the art.

The death receptor agonist and the Egr-1 antagonist of the invention can be administered sequentially, separately or combined. The most suitable route of administration can thereby easily be determined by the skilled person and is dependent on the type of antagonist used and/or the patient to be treated. For example, where the death receptor agonist and the Egr-1 antagonist are formulated as an injectable formulation, it may be desirable to choose combined administration in order to incur less stress on the patient, especially where the patient is a child. In contrast, if combined administration of the death receptor agonist and the Egr-1 antagonist, depending on the type of antagonist used, causes adverse reactions in the patient, the skilled person will want to choose separate administration.

The skilled person will also appreciate that sequential administration of the death receptor agonist and the Egr-1 antagonist is desirable if the nature of the antagonist is such that it requires some time to effect its function. For example, an antagonist that is a transcriptional antagonist may need to be administered some time before the death receptor agonist in order for the antagonist to exercise its full effect by the time the death receptor agonist is administered. Likewise, an antagonist that works by inhibiting the biological function of Egr-1 may take some time to work to its full effect.

The exact time point at which the Egr-1 antagonist should be administered before administration of the death receptor agonist varies with the type of death receptor agonist and the type of Egr-1 antagonist used. However, the best time point can be easily determined by the person skilled in the art. For example, when the Egr-1 antagonist is a transcriptional inhibitor, it is possible to administer the antagonist and then take samples from the treated subject at regular intervals in order to determine when the antagonist exerts its maximal function, i.e. at which time point the transcription of Egr-1 is lowest, as determined by standard laboratory techniques like real-time PCR etc. Likewise, when the Egr-1 antagonist works by repressing the biological function of Egr-1 samples can be taken from the treated subject at various time points after administration of the antagonist. The samples can then be used in a biological assay to determine at which time point the biological function of Egr-1 is at its lowest.

While it may be necessary to determine the ideal time point for administration of the Egr-1 antagonist for each antagonist individually, the skilled person will understand that this will only have to be determined during animal or clinical trials. Once the ideal time frame has been established, it can be assumed that this time frame will be suitable for use in all patients which are treated with the specified death receptor agonist and the Egr-1 antagonist.

### Proliferative Disorders

The method of the invention may be used to treat proliferative disorders, such as neoplasia, dysplasia, and hyperplasia. In a preferred embodiment the proliferative disorder is a cancer. These include, but are not limited to, cancers of the lung, breast, prostate, bladder, kidney, ovaries and colon as well as melanoma, leukemia, multiple myeloma and gynaecological cancers. In a preferred embodiment the cancer is a colon cancer. In some embodiments, cancers which are sensitive to death receptor agonist induced apoptosis, and particularly to TRAIL induced apoptosis may be treated with the method of the present invention.

The inventors are of the belief that particularly suitable proliferative disorders which can be treated with the method of the invention are those in which Egr-1 shows at least 1.5 fold increased expression in cells affected by the proliferative disorder compared to the expression levels of Egr-1 in tissue unaffected by the proliferative disorder from the same subject. Ways of measuring the expression level of Egr-1 are thereby well known to those of skill in the art and include real-time PCR, Northern blot analysis, semiquantitative PCR etc. In certain embodiments the expression level in the cells affected by the proliferative disorder is increased by 2fold, 3fold, 4fold, 5fold, 10fold, 100fold or even 1000fold compared to the expression levels in tissues that are unaffected by the proliferative disorder. Such proliferative disorders are considered particularly suitable, as the inventors have shown that upregulation of Egr-1 in the cell results in upregulation of the anti-apoptotic protein c-FLIP (see FIG. 4). The inventors hypothesize that this upregulation prevents apoptosis induction in the cell and may be circumvented by an Egr-1 antagonist.

### Pharmaceutical Compositions

In a further embodiment, the invention provides a pharmaceutical composition comprising a death receptor agonist and an antagonist of Egr-1, optionally in conjunction with a pharmaceutically-acceptable carrier. In one embodiment the death receptor agonist may be a member of the Tumor Necrosis Factor family, and in another embodiment the death receptor agonist may be TRAIL, TNF or fas ligand.

The death receptor agonist and/or the antagonist of Egr-1 represent the active ingredient in the composition, and this is present at a therapeutically effective amount e.g. an amount sufficient to induce apoptosis or increase the apoptosis rate. The precise effective amount for a given patient will depend upon their size and health, the nature and extent of infection, and the composition or combination of compositions selected for administration. The effective amount can be determined by routine experimentation and is within the judgement of the clinician. For purposes of the present invention, a suitable dose should be used so as to achieve a serum concentration of death receptor agonist of between 0.1 and 1000 ng/ml, between 1 ng/ml and 100 ng/ml, or around 10-100 ng/ml. The Egr-1 antagonist may be administered such that a serum concentration of between 10 nM and 20 μM, between 50 nM and 10 μM, between 100 nM and 1 μM or 200 nM to 800 nM is achieved. One or more of the active ingredients may be included in the composition in the form of salts and/or esters.

The carrier can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles. Liposomes are suitable carriers. A thorough discussion of pharmaceutical carriers is available in Gennaro ((2000) Remington: The Science and Practice of Pharmacy 20th ed, ISBN: 0683306472).

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be lyophilised.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe.

The pharmaceutical composition of the invention may contain additional components. In one embodiment the pharmaceutical composition may be co-administered with one or more other compounds, preferably antitumour compounds, more preferably those which are active against the cancerous cells targeted by the variants of the invention or those which increase responsiveness of the tumour to the death receptor agonist variants. This can be particularly important when using TRAIL variants. Compositions of the invention may thus include one or more antitumour agents, examples of which will be known to those of skill in the art and include gamma irradiation as well as chemotherapeutic drugs such as alkylating agents, anti-metabolites, plant alkaloids and terpenoids, vinca alkaloids, podophyllotoxin derivatives, taxanes, topoisomerase inhibitors, antitumour antibiotics, monoclonal antibodies, DNA damaging drugs, histone deacetylase inhibitors, hormones, proteasome inhibitors and so on.

### Targeting to the Tumour

The pharmaceutical composition comprising the death receptor agonist and/or the antagonist of Egr-1 may be delivered by any suitable route. For example, the pharmaceutical composition may be administered parenterally and may be delivered by an intravenous, rectal, oral, auricular, intraosseous, intraarterial, intramuscular, subcutaneous, cutaneous, intradermal, intracranial, intratheccal, intraperitoneal, topical, intrapleural, intra-orbital, intra-cerebrospinal fluid, transdermal, intranasal (or other mucosal), pulmonary, inhalation, or other appropriate administration route. The pharmaceutical composition may be administered directly to the desired organ or tissue or may be administered systemically. In particular, preferred routes of administration include via direct organ injection, vascular access, or via intra-muscular, intravenous, oral or subcutaneous routes.

### TRAIL Variants

The method of the invention may be practised with wtTRAIL, or a fragment thereof. A preferred soluble fragment comprises the extracellular domain (e.g. residues 114-281) of TRAIL. A soluble fragment comprising amino acids 114-281 of wtTRAII is herein termed rhTRAIL. However, in an alternative embodiment the TRAIL protein used is a variant of wt TRAIL or a wtTRAIL fragment, such as the extracellular domain.

It is known that receptors DR4 and/or DR5 may be up-regulated after treatment with DNA damaging chemotherapeutic drugs. In such cells, chemotherapeutics can significantly increase the response to TRAIL-induced apoptosis. It has been suggested in the literature that DR5 is the principal receptor that transmits the death signal. However, in at least some cancer cells the apoptotic signal is primarily transmitted by DR4. Examples of such cancers include, but are not limited to, chronic lymphocytic leukaemia and mantle cell lymphoma. For this reason, having selective inducers of DR5 (TRAIL-R2) or DR4 (TRAIL-R1) signalling is of great interest. The inventors are therefore of the view that the use of receptor selective TRAIL variants could permit better therapies with higher efficacy and possibly less side effects as compared to wild-type TRAIL.

Therefore, in a preferred embodiment, the TRAIL variant used has substantially greater affinity for the death receptor 4 (TRAIL-R1) over its affinity for the death receptor 5 (TRAIL-R2). In another preferred embodiment, the TRAIL variant has substantially greater affinity for the death receptor 5 (TRAIL-R2) over its affinity for the death receptor 4 (TRAM-R1). Methods of obtaining and screening such TRAIL variants have been previously described in WO2005/056596 and GB patent applications no. 0723059.2 and 0724532.7, which are hereby incorporated by reference in their entirety and it is therefore within the means of the person skilled in the art to obtain TRAIL variants suitable for use in the method of the present invention.

Methods of identifying cancers that may benefit from the combination treatment including the receptor specific TRAIL variants are those which preferentially express the DR4 and/or DR5 receptor on their cell surface. Such cancers are readily identifiable by various means known to those of skill in the art which include, but are not limited to, immunocytochemistry with receptor specific antibodies, Fluorescent-activated cell sorting (FACS) with receptor specific antibodies, western blot analysis with receptor specific antibodies etc. DR4 specific antibodies can be obtained, for example, from Abcam (ab8414). DR5 antibodies are available as well (Sigma-Aldrich, D3938).

In a further preferred embodiment the TRAIL variant used has substantially greater affinity for the DR4 receptor and/or the DR5 receptor over the decoy receptor(s) DcR1 (TRAIL-R3) and/or DcR2 (TRAIL-R4). Binding of TRAIL to these decoy receptors does not induce apoptosis; on the contrary, it may actually prevent apoptosis by sequestering available TRAIL from DR4 and DR5, or by leading to NF-κB activation via DcR2 (Marsters S A et al. (1997); Curr Biol, 7: 1003-1006.; Merino D et al. (2006); Mol Cell Biol, 26: 7046-7055; Pan G et al. (1997); Science, 277: 815-818.).

For this reason, it is preferred that the TRAIL variants of the invention are not sequestered via this route. Therefore, TRAIL variants which do not bind the decoy receptors or which bind to the decoy receptors with lower affinity will be more effective in inducing apoptosis as all available TRAIL proteins will bind to the apoptosis inducing receptors.

By “substantially greater affinity” we mean that there is a measurably higher affinity of the TRAIL variant for one receptor as compared with another receptor. In one embodiment, the affinity is at least 1.5-fold, 2-fold, 5-fold, 10-fold, 100-fold, or even 1000-fold or greater for one receptor compared with one or more other receptors. Methods for measuring the binding affinity of proteins for binding partners are well known in the art, including for example, competition assays, Surface Plasmon Resonance and so on.

Suitable examples of TRAIL variants with preferential binding characteristics which can be used in the method of the invention are G131R, G131K, R1491, R149M, R149N, R149K, S159R, Q193H, W193K, K201R, K204E, K204D, K204L, K204Y, K212R, S215E, N199R/K201H, S215H, S215K, S215D, D218H, K251D, K251E, K251Q, D269H, E195R, N199H/K201R, G131R/N199R/K201H, G131R/N199R/K201H, G131R/D218H, D269H/E195R, T214R and D269H/T214R. Preferably, a TRAIL variant according to the invention is a fragment comprising or consisting of residues 114-281, containing one or more of the mutations listed above.

It is also envisioned to use two or more TRAIL variants or a combination of wt TRAIL and one or more TRAIL variants in the method of the invention.

### Other Death Receptor Agonist Variants

The method of the invention may be practiced with any wild-type death receptor agonist or a fragment thereof. Particularly preferred fragments may include soluble portions of the death receptor agonist or the extra-cellular portion of the death receptor agonist.

Death receptor variants may differ from the wild type death receptor agonist sequence at one or more amino acid positions.

In one embodiment the death receptor agonist variant may be a TNF variant. The TNF variant may be selective for TNFR-I (TNFR55), such mutants may include R32W, R32W-S86T, or E146K.

In another embodiment the death receptor agonist variant may be a Fas ligand variant. The fas ligand variant may have increased affinity for Fas and may vary at one or more positions from wild type Fas ligand.

### Fusion Proteins

The death receptor agonist and/or Egr-1 antagonist (when the antagonist is a polypeptide) used in the method of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the death receptor agonist or Egr-1 antagonist may be fused with another compound, such as a compound to increase the half-life of the protein (for example, polyethylene glycol). In one embodiment the death receptor agonist may be fused to another death receptor agonist. In particular, a non-TRAIL death receptor agonist may be fused to a TRAIL death receptor agonist.

Fusion proteins that enhance the biological activity of the death receptor agonists, such as a death receptor agonist conjugated to the antimelanoma antibody ZME-018 may be used. A particular example of this may be recombinant human TNF conjugated to the anti-melanoma antibody ZME-018 (Rosenblum M G et al. Cancer Immunol Immunother. 1995 May; 40(5):322-8). A death receptor agonist may alternatively be conjugated to any one of several tumor antigens including gp240, EGFR (epidermal growth factor), Her2/Neu or single stranded DNA released from necrotic tumor cells (Christ O et al., Clin Cancer Res. 2001 May; 7(5):1385-97; Rosenblum M G et al., Int J. Cancer. 2000 Oct. 15; 88(2):267-73; Sharifi J et al., Hybrid Hybridomics. 2002 December; 21(6):421-32).

A death domain pro-drug molecule also falls within the definition of a “variant”, and a particular example of this is the TNF pro-drug molecule, which is comprised of a single chain antibody targeting fibroblast activation protein (FAP), a trimerization domain, TNF and a TNF-R1 cap separated from TNF by a protease sensitive linker (Wuest T et al., Oncogene. 2002 Jun. 20; 21(27):4257-65).

Fusion proteins between death receptor agonists and an antibody that specifically recognizes tumor cells or tumor stroma, such as the anti-CD20 antibody or the single chain antibody that specifically recognizes the tumor stroma marker FAP are also envisaged. These have been emplified with fas ligand (for review: Papenfuss et al., J. Cell Mol. Med. 2008 (6B):2566-85).

Fusion proteins can be obtained by cloning a polynucleotide encoding the protein in frame to the coding sequences for a heterologous protein sequence.

The term “heterologous”, when used herein, is intended to designate any polypeptide other than a death receptor agonist or an Egr-1 antagonist according to the invention. Examples of heterologous sequences, that can be comprised in the fusion proteins connected either at the N- or C-terminus, include: extracellular domains of membrane-bound protein, immunoglobulin constant regions (Fc regions), multimerization domains, domains of extracellular proteins, signal sequences, export sequences, tumour targeting peptides and sequences allowing purification by affinity chromatography. In the case of the Egr-1 antagonist, a fusion protein comprising a nuclear localization signal may be preferred if the antagonistic function of the protein requires the presence of the protein within the cell.

Many of these heterologous sequences are commercially available in expression plasmids since these sequences are commonly included in fusion proteins in order to provide additional properties without significantly impairing the specific biological activity of the protein fused to them (Terpe K (2003), Appl Microbiol Biotechnol, 60:523-33). Examples of such additional properties are a longer lasting half-life in body fluids, the extracellular localization, or an easier purification procedure as allowed by the a stretch of histidines forming the so-called “histidine tag” or by the “HA” tag, an epitope derived from the influenza hemagglutinin protein (Gentz et al. (1989), Proc Natl Acad Sci USA 86, 821-824). If needed, the heterologous sequence can be eliminated by a proteolytic cleavage, for example by inserting a proteolytic cleavage site between the protein and the heterologous sequence, and exposing the purified fusion protein to the appropriate protease. These features are of particular importance for the fusion proteins since they facilitate their production and use in the preparation of pharmaceutical compositions. For example, the protein may be purified by means of a hexa-histidine peptide fused at the C-terminus. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of the substances of the invention and the immunoglobulin sequence. The resulting fusion protein has improved properties, such as an extended residence time in body fluids (i.e. an increased half-life), increased specific activity, increased expression level, or the purification of the fusion protein is facilitated.

In one embodiment, the protein is fused to the constant region of an Ig molecule. Examples of Ig molecules include heavy chain regions, like the CH2 and CH3 domains of human IgG1. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

In a further preferred embodiment, the protein may comprise at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

### Other Genes Found in the Screen

In the microarray screen, carried out by the inventors, several other proteins have been identified which are differentially expressed following exposure of the cells to TRAIL, and potentially following exposure to other death receptor agonists.

In particular, the NFκB inhibitors NFκBIA and NFκBIZ were shown to be upregulated in response to treatment with TRAIL. Previous studies have shown that inhibition of NFκB increases TRAIL-mediated apoptosis (Ricci M S et al. (2007); Cancer Cell, July; 12(1):66-80). Therefore, it is likely that a combination therapy comprising an agonist that can stabilize or increase the expression of NFKBIA (NF-κB inhibitor alpha, also known as inhibitor kappa B alpha (IκBα) and/or NFKBIZ (nuclear factor of kappa light polypeptide gene enhancer in B-cells inhibitor zeta, also known as inhibitor kappa B zeta (IκB-ζ) should increase the apoptosis rate of tumours.

NFKBIA/IκBα is a ubiquitously expressed inhibitor of NF-κB and it interacts preferentially with p65/p50 and c-Rel/p50 heterodimers of the NF-κB family (Gilmore, T. D. (1999); Oncogene 18, 6842-6844). NFKBIA/IκBα is rapidly degraded upon a range of stimuli, leading to an immediate but transient activation of NF-κB.

NFKBIZ/IκB-ζ on the other hand is barely detectable in resting cells but strongly induced for example upon stimulation of the innate immune system. NFKBIZ/IκB-ζ accumulates in the nucleus, where it can associate with NF-κB subunits and regulate their transcriptional activity both positively and negatively depending on genes (Muta T. (2006); Vitam Horm.; 74:301-16.)

Assays suitable to find antagonists of Egr-1, as described earlier, may be adjusted by the skilled person to identify agonists of NFKBIA and NFKBIZ. Accordingly, one embodiment of the invention provides a method of treating a proliferative disorder in a patient comprising administering to the patient a combination of a death receptor agonist and an agonist of NFKBIA and/or NFKBIZ, particularly a combination of a TRAIL protein and an agonist of NFKBIA and/or NFKBIZ, wherein said TRAIL protein and said agonist may be for sequential, separate or combined administration. In one embodiment the TRAIL protein may be a TRAIL variant. Suitable TRAIL variants for use in this embodiment have already been discussed earlier. In a further embodiment the proliferative disorder is characterized by at least 1.5-fold increased expression of NFκB in cells affected by the proliferative disorder compared to the expression levels of NFκB in cells unaffected by the proliferative disorder from the same subject. In a further embodiment the proliferative disorder which is to be treated is a cancer. The cancer may be selected from the group consisting of cancers of the lung, breast, prostate, bladder, kidney, ovarian, colon, rectal, melanoma, leukemia, multiple myeloma and gynaecological cancers. In further embodiments the invention provides a pharmaceutical composition comprising a death receptor agonist and an agonist of NFKBIA and/or NFKBIZ. Alternatively, a proliferative disorder can be treated by overexpression of NFKBIA and/or NFKBIZ or administration of polypeptide or peptide fragments of these molecules which retain the NF-kB binding and inhibitory ability in combination with a death receptor agonist, and particularly in combination with TRAIL.

The inventors have also shown that NKD2, VDAC3 and TEAD are down-regulated following TRAIL treatment, and potentially following treatment with other death receptor agonists. Accordingly, one embodiment of the invention provides a method of treating a proliferative disorder in a patient comprising administering to the patient a combination of a death receptor agonist and an agonist of NKD2, VDAC3 and/or an antagonist of TEAD, wherein said death receptor agonist protein and said NKD2, VDAC3 and/or an antagonist of TEAD may be for sequential, separate or combined administration.

NKD2 is known to be a negative regulator of the canonical Wnt signalling pathway. This pathway is implicated in cell fate determination. It is known that other negative regulators of the canonical Wnt pathway, like APC, AXIN1, and AXIN2, are downregulated in carcinogenesis. It is therefore reasonable to assume that downregulation of NKD2 also has implications in cancer. Therefore, one embodiment of the invention provides a method of treating a proliferative disorder in a patient comprising administering to the patient a combination of a death receptor agonist protein and an agonist of NKD2, wherein said TRAIL protein and said NKD2 agonist may be for sequential, separate or combined administration. Assays suitable to find antagonists of Egr-1, as described earlier, may be adjusted by the skilled person to identify agonists of NKD2. In one embodiment the death receptor agonist may be a variant, and particularly said TRAIL protein may be a TRAIL variant. Suitable TRAIL variants for use in this embodiment have already been discussed earlier. In a further embodiment the proliferative disorder is characterized by at least 1.5-fold decreased expression of NKD2 in cells affected by the proliferative disorder compared to the expression levels of NKD2 in cells unaffected by the proliferative disorder from the same subject. In a further embodiment the proliferative disorder which is to be treated is a cancer. The cancer may be selected from the group consisting of cancers of the lung, breast, prostate, bladder, kidney, ovarian, colon, rectal, melanoma, leukemia, multiple myeloma and gynaecological cancers. In further embodiments the invention provides a pharmaceutical composition comprising a death receptor agonist and an agonist of NKD2.

VDAC3 (voltage-dependent anion channel 3) is an anion channel protein which can be found in the outer mitochondrial membrane. VDAC3 is involved in apoptogenic cytochrome c release caused by proapoptotic members of the Bcl-2 family such as Bax and Bak. Therefore, the inventors' finding that VDAC3 is downregulated in response to TRAIL is important as fewer channels in the mitochondrial outer membrane may counteract initiation of apoptosis. It may therefore be desirable to administer a death receptor agonist together with an agonist of VDAC3 expression in order to increase the efficiency of death receptor agonist treatment. Assays suitable to find antagonists of Egr-1, as described earlier, may be adjusted by the skilled person to identify agonists of VDAC3. Accordingly, one embodiment of the invention provides a method of treating a proliferative disorder in a patient comprising administering to the patient a combination of a death receptor agonist, and particularly a TRAIL protein, and an agonist of VDAC3, wherein said death receptor agonist protein and said VDAC3 agonist may be for sequential, separate or combined administration. In one embodiment the death receptor agonist may be a variant, and particularly said TRAIL protein may be a TRAIL variant. Suitable TRAIL variants for use in this embodiment have already been discussed earlier. In a further embodiment the proliferative disorder is characterized by at least 1.5-fold decreased expression of VDAC3 in cells affected by the proliferative disorder compared to the expression levels of VDAC3 in cells unaffected by the proliferative disorder from the same subject. In a further embodiment the proliferative disorder which is to be treated is a cancer. The cancer may be selected from the group consisting of cancers of the lung, breast, prostate, bladder, kidney, ovarian, colon, rectal, melanoma, leukemia, multiple myeloma and gynaecological cancers. In further embodiments the invention provides a pharmaceutical composition comprising a death receptor agonist and an agonist of VDAC3.

TEAD1 (TEF1) is a member of the TEAD family of transcription factors known for their role in expression of oncogenic viruses SV40 and HPV16 (Ishiji T et al., (1992) EMBO J.; 11(6):2271-81.). The TEAD family was identified in cancer cells and recent findings indicate that TEAD proteins, especially TEAD1 have aberrant activity in tumour tissues (Hucl T et al. (2007); Cancer Res.; 67(19):9055-65.) Recent studies have suggested that TEAD is involved in mediating transcription of YAP, a known oncogene which is amplified in human cancers. TEAD1 is also required for YAP-induced cell growth, oncogenic transformation, and epithelial-mesenchymal transition.

Given the known implication of TEAD1 in the oncogenic phenotype mediated by YAP or other factors, it may therefore be beneficial to administer antagonists of TEAD in combination with a death receptor agonist in order to enhance the efficiency of the treatment. Accordingly, one embodiment of the invention provides a method of treating a proliferative disorder in a patient comprising administering to the patient a combination of a death receptor agonist and an antagonist of TEAD1, wherein said death receptor agonist and said TEAD1 antagonist may be for sequential, separate or combined administration. Assays suitable to find antagonists of Egr-1, as described earlier, may be adjusted by the skilled person to identify antagonists of TEAD1. In one embodiment the death receptor agonist may be a variant, and in particular said TRAIL protein may be a TRAIL variant. Suitable TRAIL variants for use in this embodiment have already been discussed earlier. In a further embodiment the proliferative disorder is characterized by at least 1.5-fold increased expression of TEAD1 in cells affected by the proliferative disorder compared to the expression levels of TEAD1 in cells unaffected by the proliferative disorder from the same subject. In a further embodiment the proliferative disorder which is to be treated is a cancer. The cancer may be selected from the group consisting of cancers of the lung, breast, prostate, bladder, kidney, ovarian, colon, rectal, melanoma, leukemia, multiple myeloma and gynaecological cancers. In further embodiments the invention provides a pharmaceutical composition comprising a death receptor agonist, particularly a TRAIL protein, and an antagonist of TEAD1.

Various aspects and embodiments of the present invention will now be described in more detail by way of example. It will be appreciated that modification of detail may be made without departing from the scope of the invention.

### BRIEF DESCRIPTION OF DRAWINGS

FIG. 1 Induction of Egr-1 following treatment with rhTRAIL.

(A) Validation of cDNA microarray results. Colo205 cells were treated with 10 ng/ml of rhTRAIL and total RNA was isolated at the times indicated. mRNA expression level of TEAD1, VDAC3, NKD2, Egr-1, c-Jun, NFKBIA and NFKBIZ were assessed by RTPCR. GAPDH was used as internal control. The figure shows one representative picture of three independent experiments. (B) Induction of Egr-1 protein by TRAIL receptor activation. Colo205, HCT15 and HCA7 cells were treated with rhTRAIL at 10 ng/ml (Colo205) and 50 ng/ml (HCT15 and HCA7) concentrations. Cell lysates were prepared at the indicated times and analysed by Western blotting for the expression level of Egr-1. Actin expression was used as a loading control. The figure shows representative images of two independent experiments.

FIG. 2 Dominant negative Egr-1 potentiates rhTRAIL-induced apoptosis.

(A) Overexpression of a dominant negative Egr-1 (EBGN-EGR-1) protein in HCT15 cells. HCT15 cells were transiently transfected with EBGN-EGR-1 or empty vector. Cell lysates were analysed for overexpression of EBGN-EGR-1 24 h post-transfection by Western blotting.

FIG. 3 TRAIL-mediated apoptosis does not require mitochondrial amplification in HCT15 cells

(A) Bcl-2 expression in Bcl-2 overexpressing HCT15 cells. HCT15 cells were stably transfected with mitochondrial localized Bcl-2 (mt-Bcl-2) expressing plasmid or empty vector. Overexpression mt-Blcl-2 was confirmed by Western blotting. (B) Effect of Bcl2 overexpression on TRAIL-induced HCT15 apoptosis. Mock- and Bcl-2-transfected HCT15 cells were treated with 50 ng/ml of TRAIL for 12 h and apoptosis was assessed by Annexin V staining. Results are presented as means±S.E.M. of three independent experiments.

FIG. 4 EBGN-EGR-1 reduces c-FLIP expression in HCT15 cells

(A) Cell lysates were prepared from HCT15 cells transiently transfected with dominant negative Egr-1 expressing plasmid or empty vector at 24 h post-transfection. Basal levels of c-FLIPL and c-FLIPS are shown measured by Western blot analysis. Actin expression was determined to serve as loading control. (B) Densitometric quantification of c-FLIPL and c-FLIPS blots using GeneTools software (version 3.07, Syngene). c-FLIP expression values were normalized to actin expression level. Results are presented as mean±S.E.M. of 3 independent experiments,* p<0.05. (C) Knockdown of c-FLIP potentiates TRAIL induced apoptosis. HCT15 cells were nucleofected with 3 different siRNAs targeting c14 FLIP (c-FLIP1-3). 24 h post-transfection cells were treated with 50 ng/ml rhTRAIL for 3 h and apoptosis induction was measured by Annexin V assay. siRNA against GFP (green fluorescent protein) was used as a negative control. The graph is a representative of two independent experiments.

FIG. 5 Knockdown of Egr-1 enhances TRAIL-induced apoptosis

HCT15 cells were transiently transfected with a Smartpool siRNA mix against Egr-1 (Egr-1) or non-target siRNA (control siRNA, C). (A) Knockdown of Egr-1 was confirmed 24 h post-transfection by Western blotting. (B) Non-target siRNA transfected (C) and Egr-1 siRNA transfected (Egr-1) cells were treated at 24 h post-transfection with 50 ng/ml rhTRAIL for 4 h and induction of apoptosis was assessed by Annexin V staining. Results are presented as means±S.E.M. of percentage apoptosis induced; * Significant difference with p<0.05.

FIG. 6 Egr-1 drives c-FLIP expression

HCT15 cells were transiently transfected with a Smartpool siRNA mix against Egr-1 (Egr-1) or non-target siRNA (control siRNA, C). (A) Egr-1 knockdown reduces expression of c-FLIP_{L} and c-FLIPₛ examined in whole cell lysates 24 h post-transfection of the siRNAs by Western blotting. Actin expression levels were detected as a loading control and Egr-1 expression levels were detected to monitor knockdown efficiency. (B) Densitometric quantification of c-FLIP_{L} and c-FLIP_{S} levels. The graph shows averaged band densities normalised for β-actin levels in whole cell lysates from three independent experiments.

FIG. 7 Inhibition of Egr-1 increases TNF- and anti-Fas antibody-induced cell death

HCT15 cells were transfected with empty vector (EV) or DN-Egr-1 before treatment with rhTRAIL (100 ng/ml), rhTNF (60 ng/ml) or agonistic anti-Fas antibody (100 nM) for 4 h. Induction of apoptosis was determined on cytospins stained with hematoxylin-eosin by counting 300 cells/slide.

### EXAMPLES

### Example 1

### Tissue Culture

Colo205 cells were obtained from American Tissue Culture Collection (ATCC). HCT15 and HCA7 cells were a kind gift from Prof. L. Egan (University College Hospital, Galway). Colo205 and HCT15 cells were maintained in RPMI-1640 medium and HCA7 in DMEM medium, both media supplemented with 10% foetal bovine serum (FBS), 2 mM glutamine, 50 U/ml penicillin and 50 mg/ml streptomycin at 37° C., 5% CO₂ in a humidified incubator. Cells were seeded at 2×10⁵ cells/ml one day prior to treatment. To induce apoptosis, cells were treated with rhTRAIL (non-tagged, fragment amino acids 114-281, Triskel Therapeutics, Groningen) and DR5-selective mutants D269H, D269H-E195R and D269H-T214R and agonistic DR4 or DR5 antibodies (Novartis Pharmaceuticals) at the concentration and times specified in the figure legends. All reagents were from Sigma-Aldrich unless otherwise stated.

### Example 2

### Differential Expression of Genes Following Exposure of Cells to rhTRAIL and DR5-Selective TRAIL

To generate a profile of genes differentially regulated by TRAIL-receptors, Colo205 cells were treated with either rhTRAIL or the DR5-selective rhTRAIL variants D269H and D269H/E195R for 1 h. rhTRAIL is a soluble fragment template comprising amino acids 114-281 of wtTRAIL (accession number NM_{—}003810.2, see also GB0724532.7 and GB0723059.2). Total RNA was isolated from these cells using GenElute RNA miniprep kit as per manufacturer's protocol. Reverse transcription (RT) was carried out with 2 μg RNA using Oligo(dT) primers (Invitrogen) and AMV Reverse Transcriptase. The cDNA product was subjected to 25-30 cycles of PCR using primers specific for Egr-1, c-Jun, TEAD-1, NKD2 VDAC3, NFKBIA and NFKBIZ. For normalisation, GAPDH PCR was carried out. The primers used for the PCR reactions were as follows:

| Gene name | Reverse sequence | Forward sequence |
|---|---|---|
| Egr-1 | 5′-AAGAACTTGGACATGG | 5′-GAAAGAAAGGGAAAAGGC |
| | CTGTTT | AGAA |
| c-Jun | 5′-CCTGACCATAGCATCA | 5′-ACTCCCCTAACCTGTTTT |
| | AGTACA | CTGC |
| TEAD1 | 5′-AACTTTGGTGGAACAG | 5′-CATTGCTTGAATCAGTGG |
| | GTGACT | ACAT |
| VDAC3 | 5′-TAGACTTCAGTGTGGG | 5′-GGAAGCTTAATGTGGTTT |
| | AGGAT | GAGG |
| NEκBIA | 5′-TCCATCTTGAAGGCTA | 5′-GCCCTGGTAGGTAACTCT |
| | CCAACT | GTTG |
| NEκBIZ | 5′-CTGTCTTTTGTGAATG | 5′-GAGCTCGCTGCTGAATGG |
| | CAAAGG | ACTT |
| NKD2 | 5′-CGGCAGGTAGTAGCTG | 5′-AGATACACATGCCGTACA |
| | AAGG | CCAC |
| GAPDH | 5′-TCCACCACCCTGTTGC | 5′-ACCACAGTCCATGCCATC |
| | TG | |

Microarray hybridization and bioinformatics analysis was carried out by ArraDx Array Based Diagnostics using Affymetrix human HgU133 Plus 2.0 GeneChips in triplicate. Single-channel experiments were carried out with all RNA samples labelled with biotin. Briefly, double stranded cDNA was synthesized from 5 μg total RNA, purified and biotin labelled. Labelled cDNA was fragmented, purified and quantified prior to its hybridization to Affymetrix human HgU133 Plus 2.0 gene chips for 16 h at 45° C. The arrays were washed, stained with Streptavidin Phycoerythrin solution for 10 min at 25° C., re-washed and probed with a biotinylated antibody solution for 10 min at 25° C. The Streptavidin Phycoerythrin solution was added for a further 10 min and washed prior to scanning. The GeneSpring data analysis program (Silicon Genetics/Agilent) was used for bioinformatic assessment. Fold increases or decreases induced were compiled for the treatments. Genes with greater then a 2-fold change and a t-test p value less then 0.05 were considered significant.

The analysis revealed 69 genes, which were differentially expressed by at least one treatment. Cluster analysis identified four genes regulated by both TRAIL and DR5-selective variants, namely CDC42 effector protein 1 (CDC42EP1), early growth response-1 (Egr-1), TEA domain family member 1 (TEAD1) and voltage gated anion channel 3 (VDAC3). Egr-1 was represented by two spots, while TEAD1 was represented by three spots on the microarray. These replicate spots showed the same regulatory pattern confirming that these genes are common and early targets of the TRAIL signalling pathway. Genes were grouped according to proposed protein function (Table 1A). 4 genes were involved in intracellular transport, 3 played a role in post-translational modifications, 10 were associated with transcription/translation regulation, 4 were involved in cellular proliferation, 3 were potential protein kinases or phosphatases, 2 were NFκB inhibitor proteins, the protein product of 2 were helicases and 8 were cancer related genes. Of these genes, seven candidates were selected for further analysis based on proposed biological function and fold induction/repression (Table 1B). The full list of genes differentially regulated is listed in Table 2.

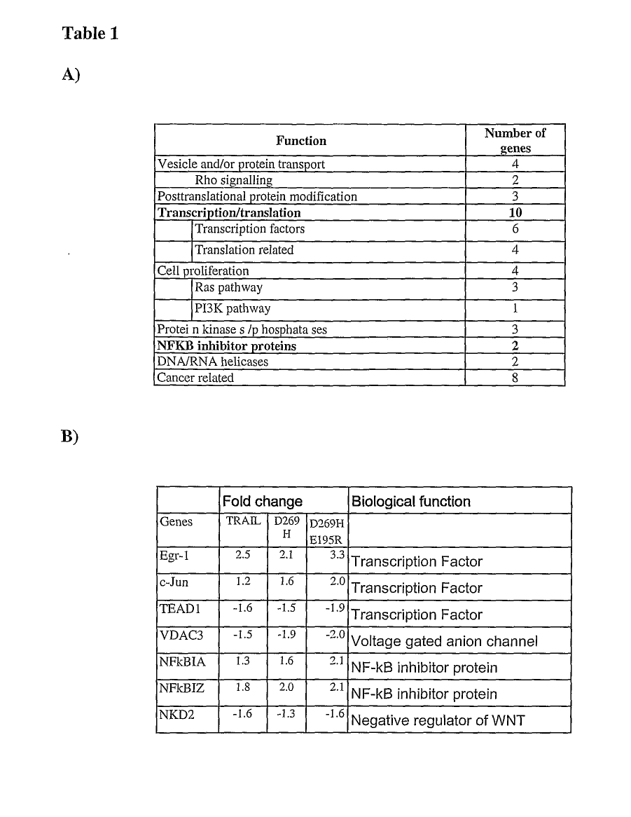

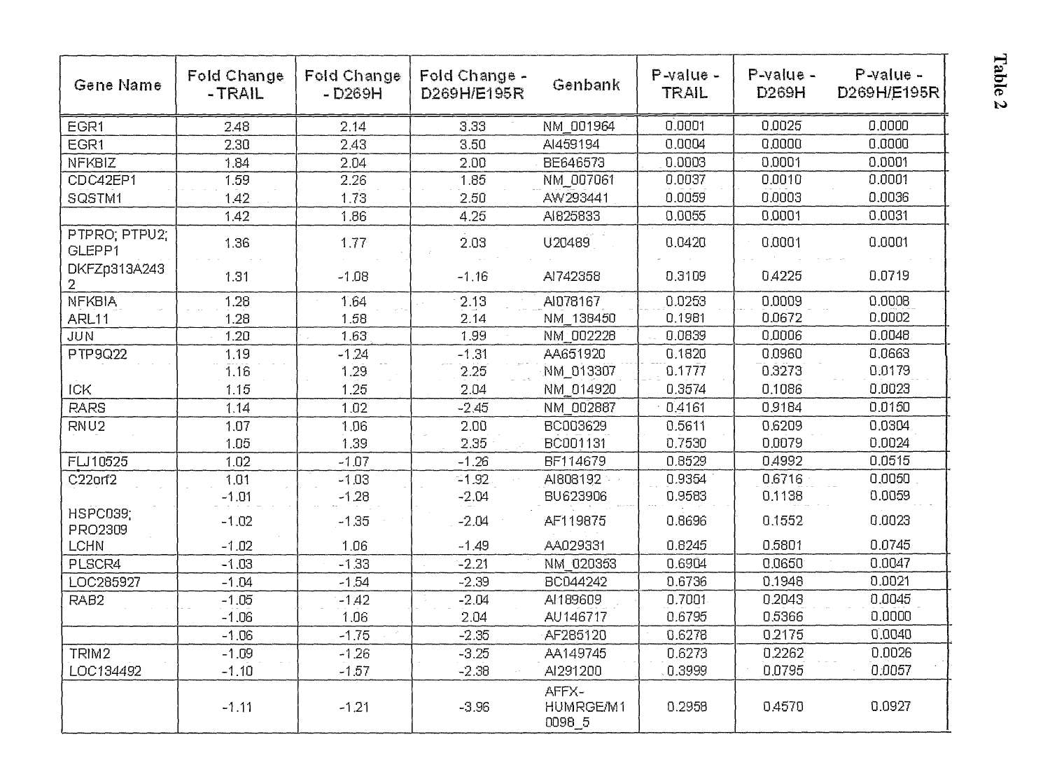

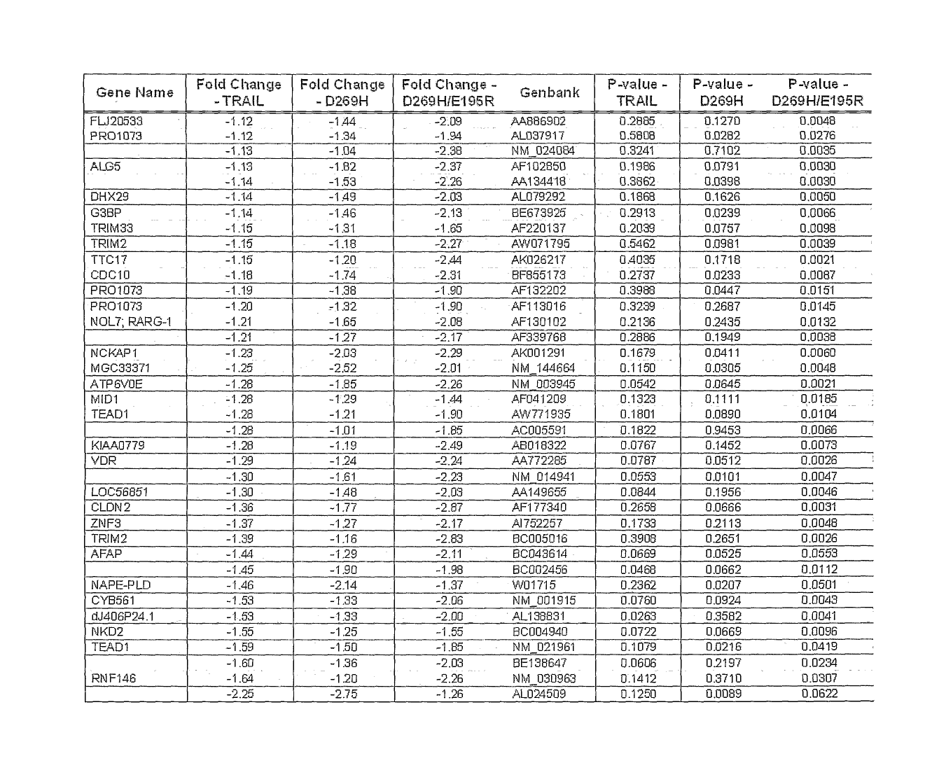

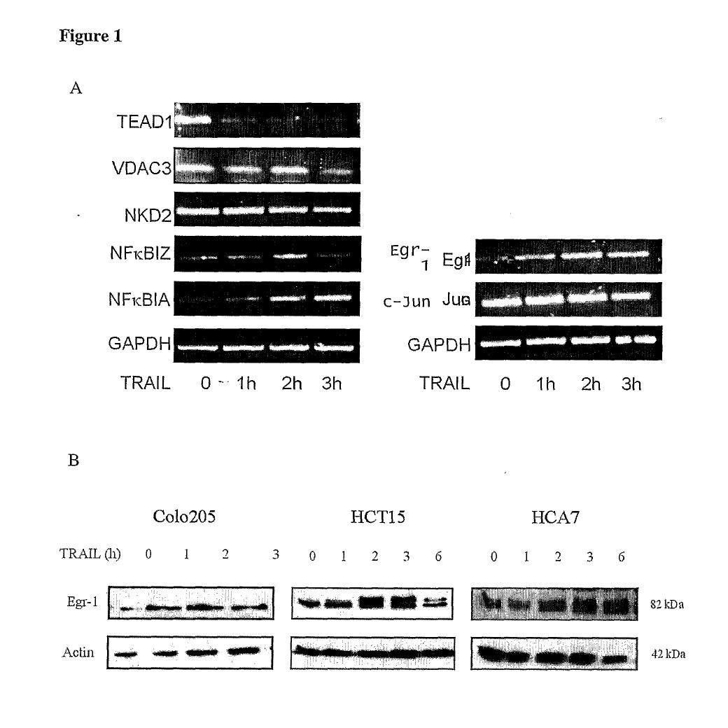

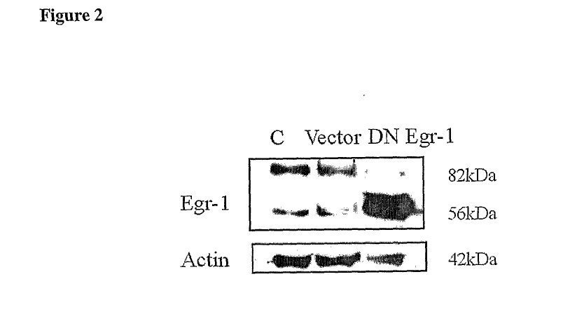

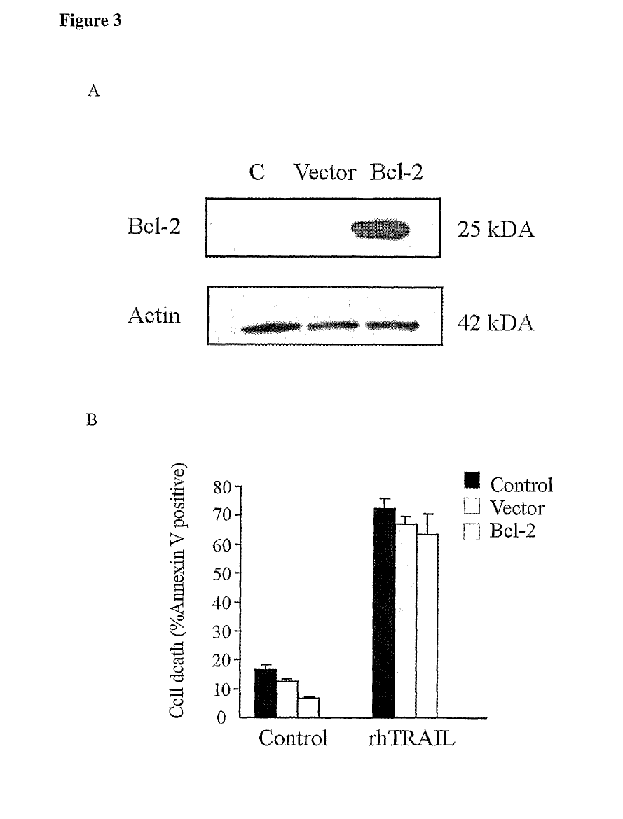

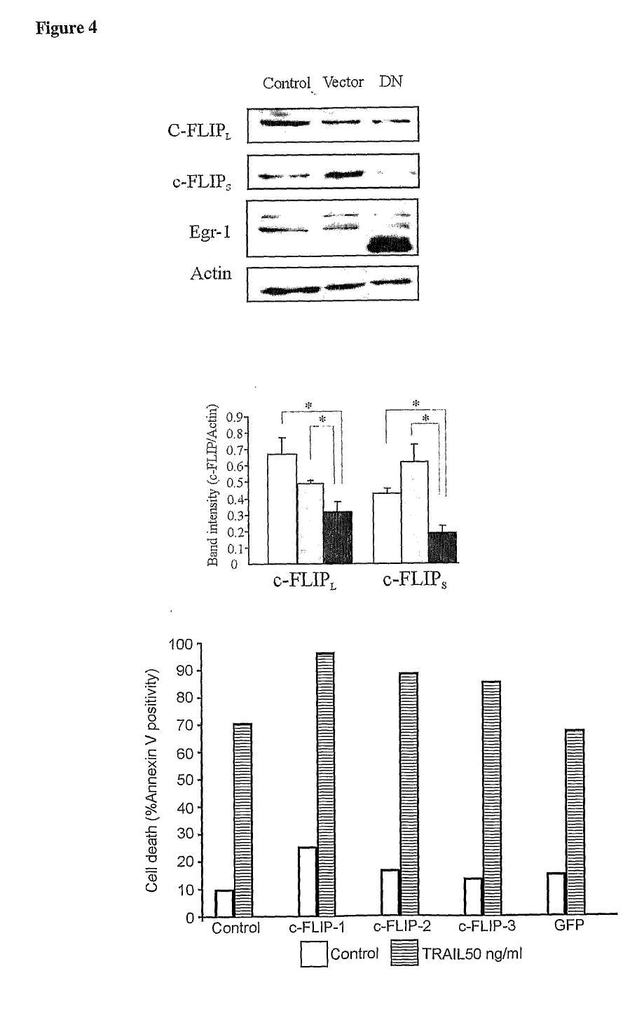

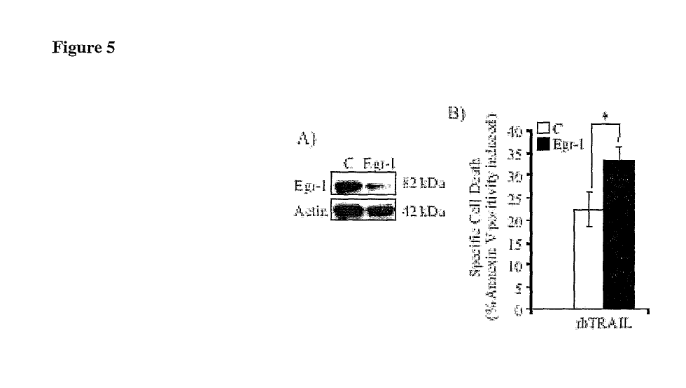

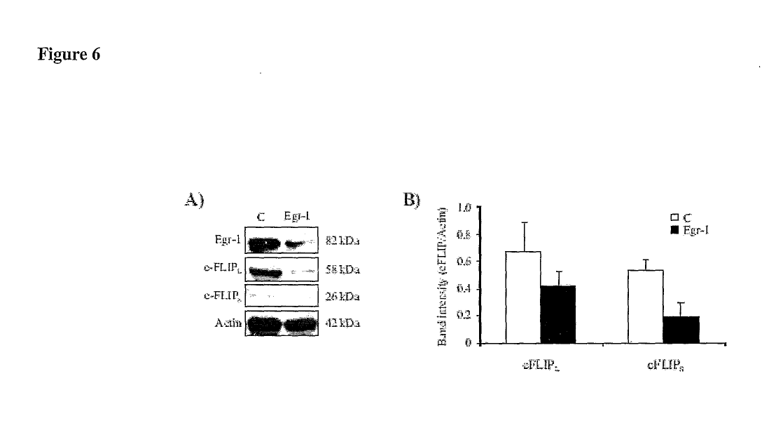

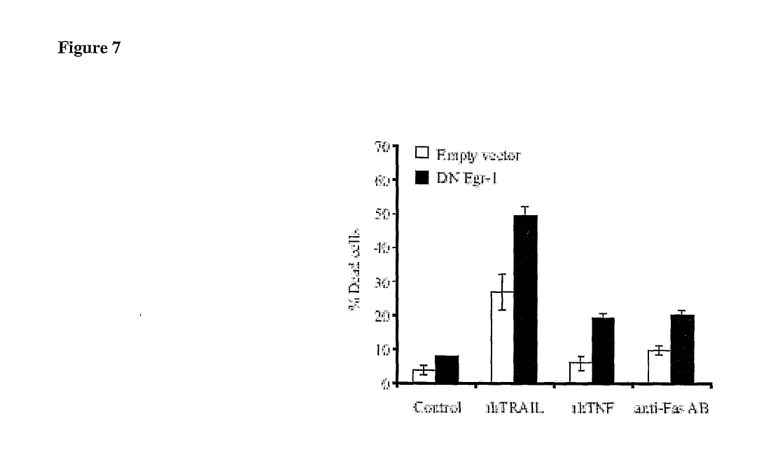

Differentially expressed genes identified with the microarray were validated by RT-PCR. Upregulation of Egr-1, NFKBIA and NFKBIZ and downregulation of NKD2, VDAC3 and TEAD in colo205 cells treated with 10 ng/mL of TRAIL or 10 ng/ml of DR5-selective rhTRAIL variants were confirmed. However, RT-PCR failed to replicate the c-Jun induction observed by microarray analysis (FIG. 1A).

Next, we focused our attention on Egr-1, a transcription factor implicated in tumour apoptosis following diverse stimuli but with limited data on its role in TRAIL-induced apoptosis. In parallel, we examined the phosphorylation status of the transcription factor c-Jun. First, we analyzed the protein expression level of Egr-1 in Colo205, HCT15 and HCA7 cell lines following treatment with rhTRAIL. Western Blot analysis confirmed induction of Egr-1 protein in Colo 205 treated with 10 ng/ml rhTRAIL and DR5-selective rhTRAIL variants (FIG. 1B). Similarly, Egr-1 induction was observed in HCT15 and HCA7 treated with 50 ng/ml rhTRAIL (FIG. 1B). Egr-1 induction was observed as early as 1 h with maximum protein observed 2-3 h post-treatment. In line with the RT-PCR results, total c-Jun was not induced at protein level in the colon cancer cell lines examined. However, in all three cell lines, c-Jun was phosphorylated in a time dependent manner by rhTRAIL (FIG. 1C).

### Example 3

### Overexpression of Dominant Negative Egr-1 Increases Apoptosis Induced by DR5

To determine whether Egr-1 plays a role in TRAIL-induced apoptosis, HCT15 cells were transiently transfected with a dominant negative Egr-1 expressing plasmid (EBGN-Egr-1, Al-Sarraj A et al. (2005); J Cell Biochem 94: 153-167), which encodes an Egr-1 mutant that lacks the trans activation domain of wild type Egr-1. Cells (2×10⁶) were pelleted and resuspended in transfection solution V (Amaxa) containing either 2.5 μg of mammalian dominant negative Egr-1 construct (EBGN-EGR-1) or empty vector (EBGN), a kind gift from G. Thiel. Similarly stable transfection of Bcl-2 plasmid or the empty vector (Neo) was generated in HCT15 cells using the similar transfection solution and stable clones generated following treatment with 1 μM of G418. Cells were transfected by nucleofection using program T13 as per manufacturer's protocol (Amaxa). GFP plasmid (2.5 μg) was also transfected into cells to determine transfection efficiencies. Control cells were subjected to similar transfection condition without any plasmids. 24 h post-transfection, cells were resuspended in media and seeded for Annexin V and protein assays.

Overexpression of dominant negative Egr-1 protein (DN-Egr-1) was confirmed by Western blot analysis (FIG. 2A). Cells were lysed in buffer containing 1% Triton X-100, 100 mM Tris/HCL pH 8.0, 200 mM sodium chloride (NaCl), 5 mM EDTA, 10% glycerol, 1 mM dithiothreitol (DTT), 1 mM phenylmethylsulphonyl fluoride (PMSF), 5 μg/ml aprotinin, 2.5 μg/ml leupeptin, 1 mM sodium orthovanadate (Na₂VO₃) and 1 mM sodium fluoride (NaF). Cellular proteins (30 μg) were separated by electrophoresis on 10% SDS polyacrylamide gel electrophoresis (SDS-PAGE) and transferred onto nitrocellulose membranes. After blocking in 5% non-fat milk and 0.05% Tween-20 in PBS, blots were incubated with rabbit monoclonal antibodies to total Egr-1 or total c-Jun (1:1,000; Santa Cruz Technologies) and mouse monoclonal antibodies to phosphorylated (p)-c-jun (1:1,000; Santa Cruz Technologies) and c-FLIP (1:500 Alexis Pharmaceutical). For detection, appropriate horseradish peroxidase-conjugated goat secondary antibodies were used. Protein bands were visualized with SuperSignal West Pico Chemiluminescent Substrate (Pierce) on X-ray film (Agfa). Stable transfectants could not be generated, suggesting a central role for Egr-1 in cell viability.

Following 5 h of treatment with 10 nM of rhTRAIL, HCT15 cells overexpressing DN-Egr-1 suffered significantly more apoptosis than untransfected cells or cells transfected with the empty vector. These data suggest that in HCT15 cells Egr-1 has an anti-apoptotic function.

Cell viability was monitored by 2-(4,5-dimethyltriazol-2-yl)-2,5-diphenyl tetrazolium bromide (MIT) assay. Following treatment, MTT (0.5 mg/ml) was added to cells and incubated for 3 h at 37° C. The reaction was stopped by addition of an MTT stop solution containing 20% SDS and 50% dimethylformamide. The purple formazan precipitate generated was allowed to dissolve for 1 h on an orbital shaker. The colour intensity was measured at 550 nm on a Wallac Victor 1420 Multilabel counter (Perkin Elmer Life Sciences). Cell viability was expressed relative to the absorbance of untreated control cells, which was taken as 100% viable.

Cell death was monitored by labelling of phosphatidyl serine externalised on the surface of apoptotic cells with Annexin-V-FITC (IQ cooperation). Following treatment, cells were collected by gentle trypsinization and incubated for 10 min at 37° C. to allow membrane recovery. Cells were pelleted by centrifugation at 350×g and incubated with Annexin-V-FITC in calcium buffer (10 mM HEPES/NaOH, pH 7.4, 140 mM NaCl and 2.5 in M CaCl₂) for 15 min on ice in the dark. Cells were washed in calcium buffer prior to acquisition on a FacsCalibur flow cytometer (Becton Dickinson). Analysis was performed using Cell Quest software (Becton Dickinson).

In order to identify the mechanism by which EBGN-EGR-1 potentiated TRAIL-induced apoptosis, it was determined whether TRAIL-induced apoptosis requires mitochondrial amplification, i.e. is it a type I or type II mechanism. Overexpression of Bcl-2 has been shown to block apoptosis in type II cells. To this end, stable Bcl-2 overexpressing HCT15 cells were generated (FIG. 3A) and treated with 50 ng/ml rhTRAIL for 12 h. Bcl-2 overexpression failed to decrease apoptosis induction by rhTRAIL (FIG. 3B), indicating that in HCT15 cells the TRAIL-induced apoptotic pathway does not require mitochondrial amplification and the likely target(s) of EBGN-EGR-1 is not a Bcl-2 protein, but a regulator of the extrinsic apoptotic pathway.

The potentiation of TRAIL-induced apoptosis by EBGN-EGR-1 in HCT15 cells could possibly be due to the repression of an intracellular-acting apoptosis inhibitory gene. As the mitochondrial pathway was not required for TRAIL-mediated apoptosis, we next examined whether overexpression of EBGN-EGR-1 can modulate the expression level of c-FLIP, an anti-apoptotic protein that affects the extrinsic death pathway only at the level of the DISC. Western blot analysis and densitometric quantitation demonstrated that overexpression of EBGN-EGR-1 decreased expression of both the long and short splice variants of c-FLIP (c-FLIP_{L} and c-FLIP_{S}, FIGS. 4A and 4B).

In order to prove that downregulation of c-FLIP, not another protein, by DN-Egr-1 resulted in enhanced TRAIL sensitivity, c-FLIP expression was downregulated by siRNA. To this end HCT15 cells (2×10⁶) were pelleted and resuspended in transfection solution V (Amaxa) containing either 2.5 μg of mammalian dominant negative Egr-1 construct (EBGN-EGR-1) or empty vector (EBGN), a kind gift from G. Thiel. Cells were transfected by nucleofection using program T13 as per manufacturer's protocol (Amaxa). GFP plasmid (2.5 μg) was used to determine transfection efficiency. Control cells were subjected to the same transfection condition without any plasmids. 24 h post-transfection, cells were resuspended in media and seeded for Annexin V and protein assays. Similarly, stable transfection of Bcl-2 or empty vector (Neo) was generated in HCT15 cells using the same transfection protocol. Pools of stable clones were selected with 1 μM of G418. siRNA transfection was carried out by the same nucleofection protocol as for plasmids using 50 nM siRNA. The following c-FLIP sequences were targeted: cFLIP1: ggagcagggacaagttaca, cFLIP2: gcaaggagaagagtttctt, cFLIP3 sense: gaggtaagctgtctgtcgg. The GFP-specific sequence was: ggcuacguccaggagcgcacc. All three siRNAs reduced c-FLIP expression, with sequence 1 (c-FLIP-1) being the most efficient. C-FLIP knockdown could potentiate TRAIL-induced apoptosis in the HCT15 cells, confirming that c-FLIP is at least one of the targets of Egr-1 through which Egr-1 controls TRAIL sensitivity (FIG. 4).

### Example 4

### Knockdown of Egr-1 by siRNA Increases Apoptosis Induced By TRAIL

The role of Egr-1 in regulating TRAIL-induced apoptosis was shown in HCT15 cells. HCT15 cells were transiently transfected with a mix of four siRNA molecules designed to silence Egr-1 expression (Smartpool, Dharmacon). Cells (2×106) were pelleted and resuspended in transfection solution V (Amaxa) containing either 50 μM of Egr-1 siRNA Smartpool or control, non-target siRNA. Cells were transfected by nucleofection using program T13 as per manufacturer's protocol (Amaxa). 24 h post-transfection, cells were seeded for treatments and harvested for Annexin V and protein assays.

Knockdown of the Egr-1 protein (DN-Egr-1) was confirmed 24 h posttransfection by Western blot analysis as described in Example 3 (FIG. 5A). Following 5 h of treatment with 10 nM of rhTRAIL, HCT15 cells transfected with Egr-1 siRNA displayed more apoptosis than cells transfected with control (non-target) siRNA. These data corroborate the finding that in HCT15 cells Egr-1 has an anti-apoptotic function (FIG. 5B).

Cell death was monitored by labelling of phosphatidyl serine externalised on the surface of apoptotic cells with Annexin-V-FITC (IQ cooperation). Following treatment, cells were collected by gentle trypsinization and incubated for 10 min at 37° C. to allow membrane recovery. Cells were pelleted by centrifugation at 350×g and incubated with Annexin-V-FITC in calcium buffer (10 mM HEPES/NaOH, pH 7.4, 140 mM NaCl and 2.5 mM CaCl₂) for 15 min on ice in the dark. Cells were washed in calcium buffer prior to acquisition on a FacsCalibur flow cytometer (Becton Dickinson). Analysis was performed using Cell Quest software (Becton Dickinson).

Egr-1 inhibits TRAIL-induced apoptosis by driving c-FLIP expression. Egr-1 expression was knocked down with siRNA in HCT15 cells as described above. Western blot analysis and densitometric quantitation demonstrated that knockdown of Egr-1 decreased expression of both the long and short splice variants of c-FLIP (c-FLIP_{L} and c-FLIP_{S}, FIGS. 6A and 6B).

### Egr-1 Regulates Sensitivity of Cancer Cells to Several Death Ligands

The following study shows that Egr-1 increases resistance of tumour cells against the death ligands Fas ligand/CD95 ligand and Tumor Necrosis Factor (TNF). As Egr-1 regulates c-FLIP expression and c-FLIP is a general inhibitor of pro-caspase-8 activation by all death receptors it was tested how inhibition of Egr-1 affects apoptosis induction by two other death receptors, TNF receptor and Fas Inhibition of Egr-1 transcriptional activity with DN-Egr-1 (as described in Example 3) increased apoptosis induction by TNF receptor and Fas in HCT15 cells (FIG. 7). Empty vector or DN-Egr-1 expressing HCT15 cells were treated with 60 ng/ml recombinant human TNF (PromoCell) or 100 nM agonistic anti-Fas antibody (clone CH-11, AMB Biotechnology) for 4 h. The cells were trypsinized and spun onto microscope slides. Induction of apoptosis was quantitated by morphological analysis of haematoxylin and eosin stained cytospins. For haematoxylin-eosin staining, the cytospins were fixed in 100% methanol for 5 min at room temperature, followed by staining with Harris haematoxylin (Sigma) for 15 minutes and Eosin Y (Sigma) for 5 minutes. Excess stain was removed by washing the slides in tap water.

1. A method of treating a proliferative disorder in a patient comprising administering to the patient a combination of an agonist of a death receptor and an antagonist of Egr-1, wherein said death receptor agonist and said Egr-1 antagonist are for sequential, separate or combined administration.

2. The method of claim 1 wherein the agonist of a death receptor is a member of the tumor necrosis factor ligand superfamily.

3. The method of claim 2 wherein the agonist of a death receptor is TRAIL, Fas ligand or TNF.

4. The method of claim 1, wherein the proliferative disorder is characterized by at least 1.5-fold increased expression of Egr-1 in cells affected by the proliferative disorder compared to the expression levels of Egr-1 in cells unaffected by the proliferative disorder from the same subject.

5. The method of claim 1, wherein the proliferative disorder is cancer.

6. The method of claim 5 wherein the cancer is selected from the group consisting of cancers of the lung, breast, prostate, bladder, kidney, ovarian, colon, rectal, melanoma, leukemia, multiple myeloma and gynaecological cancers.

7. The method of claim 1 wherein the Egr-1 antagonist is selected from the group consisting of antibodies, dominant negative Egr-1 variant expressing vectors peptides, small molecule inhibitors, RNAi (shRNA, shRNA expressing vectors, siRNA), microRNA (miRNA).

8. A pharmaceutical composition comprising a death receptor agonist and an antagonist of Egr-1.

9. The pharmaceutical composition of claim 8 wherein the death receptor agonist is a member of the tumor necrosis factor ligand superfamily.

10. The pharmaceutical composition of claim 8 wherein the death receptor agonist is TRAIL, Fas ligand or TNF.

11. The method of claim 1, wherein the death receptor agonist is a death receptor agonist variant.

12. The method of claim 11 wherein the death receptor agonist variant has substantially greater affinity for the death receptor 4 (TRAIL-R1) over its affinity for the death receptor 5 (TRAIL-R2).

13. The method of claim 11, wherein the death receptor agonist variant has substantially greater affinity for the death receptor 5 (TRAIL-R2) over its affinity for the death receptor 4 (TRAIL-R1).

14. The method of claim 11, wherein the death receptor agonist variant has substantially greater affinity for the death receptor 4 (TRAIL-R1) and/or the death receptor 5 (TRAIL-R2) over its affinity for the decoy receptor DcR1 (TRAIL-R3) and/or DcR2 (TRAIL-R4).

15. The method of claim 14 wherein the death receptor agonist variant is a TRAIL variant and wherein the TRAIL variant is selected from the group consisting of G131R, G131K, R149I, R149M, R149N, R149K, S159R, Q193H, W193K, N199R/K201H, N199H/K201R, G131R/N199R/K201H, G131R/N199R/K201H, G131R/D218H, K201R, K204E, K204D, K204L, K204Y, K212R, S215E, S215H, S215K, S215D, D218H, K251D, K251E, K251Q, D269H, E195R, D269H/E195R, T214R and D269H/T214R.

16. A kit comprising an agonist of a death receptor and an antagonist of Egr-1 for treating a proliferative disorder, wherein said death receptor agonist and said Egr-1 antagonist are for sequential, separate or combined administration.

17. The kit of claim 16, wherein the agonist is TRAIL, TNF or Fas ligand.


SEQUENCE LISTING (24 sequences)

SEQ ID NO: 1 - RNA, 21 nt, Artificial Sequence
  Synthetic siRNA molecule
uugggccaaugauggagaaua

SEQ ID NO: 2 - RNA, 21 nt, Artificial Sequence
  Synthetic siRNA molecule
uucacgacgauaguuaauaau

SEQ ID NO: 3 - RNA, 21 nt, Artificial Sequence
  Synthetic siRNA molecule
uucgagggaaguuacgaucuu

SEQ ID NO: 4 - RNA, 21 nt, Artificial Sequence
  Synthetic siRNA molecule
uucacugacaaaccgaauauu

SEQ ID NO: 5 - RNA, 21 nt, Artificial Sequence
  Synthetic siRNA molecule
uugagggaaguuacgaucuuu

SEQ ID NO: 6 - RNA, 21 nt, Artificial Sequence
  Synthetic siRNA molecule
uuccagguccuuguaacguua

SEQ ID NO: 7 - DNA, 22 bp, Artificial Sequence
  Synthetic PCR primer
aagaacttggacatggctgttt

SEQ ID NO: 8 - DNA, 22 bp, Artificial Sequence
  Synthetic PCR primer
gaaagaaagggaaaaggcagaa

SEQ ID NO: 9 - DNA, 22 bp, Artificial Sequence
  Synthetic PCR primer
cctgaccatagcatcaagtaca

SEQ ID NO: 10 - DNA, 22 bp, Artificial Sequence
  Synthetic PCR primer
actcccctaacctgttttctgc

SEQ ID NO: 11 - DNA, 22 bp, Artificial Sequence
  Synthetic PCR primer
aactttggtggaacaggtgact

SEQ ID NO: 12 - DNA, 22 bp, Artificial Sequence
  Synthetic PCR primer
cattgcttgaatcagtggacat

SEQ ID NO: 13 - DNA, 21 bp, Artificial Sequence
  Synthetic PCR primer
tagacttcagtgtgggaggat

SEQ ID NO: 14 - DNA, 22 bp, Artificial Sequence
  Synthetic PCR primer
ggaagcttaatgtggtttgagg

SEQ ID NO: 15 - DNA, 22 bp, Artificial Sequence
  Synthetic PCR primer
tccatcttgaaggctaccaact

SEQ ID NO: 16 - DNA, 22 bp, Artificial Sequence
  Synthetic PCR primer
gccctggtaggtaactctgttg

SEQ ID NO: 17 - DNA, 22 bp, Artificial Sequence
  Synthetic PCR primer
ctgtcttttgtgaatgcaaagg

SEQ ID NO: 18 - DNA, 22 bp, Artificial Sequence
  Synthetic PCR primer
gagctcgctgctgaatggactt

SEQ ID NO: 19 - DNA, 20 bp, Artificial Sequence
  Synthetic PCR primer
cggcaggtagtagctgaagg

SEQ ID NO: 20 - DNA, 22 bp, Artificial Sequence
  Synthetic PCR primer
agatacacatgccgtacaccac

SEQ ID NO: 21 - DNA, 18 bp, Artificial Sequence
  Synthetic PCR primer
tccaccaccctgttgctg

SEQ ID NO: 22 - DNA, 18 bp, Artificial Sequence
  Synthetic PCR primer
accacagtccatgccatc

SEQ ID NO: 23 - protein, 281 aa, Homo sapiens [sequence omitted]

SEQ ID NO: 24 - DNA, 1776 bp, Homo sapiens
tttcatttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcaaaaaaaaaaaaaaaaaa